(19) United States
(12) Patent Application Publication
(10) Pub. No.: US 2026/0232615 A1
(43) Pub. Date: Aug. 13, 2026

(54) FENOFIBRATE-BASED OILY FORMULATION, ORAL MEDICINE, AND PREPARATION METHOD THEREOF

(71) Applicant: INNOVATION CENTER OF YANGTZE RIVER DELTA, ZHEJIANG UNIVERSITY, Zhejiang (CN)

(72) Inventors: Xiangrui LIU, Zhejiang (CN); Tianhua ZHOU, Zhejiang (CN); Yuanyuan LIN, Zhejiang (CN); Mengying WEI, Zhejiang (CN); Hongda CHEN, Zhejiang (CN); Bingxiang ZHAO, Beijing (CN)

(21) Appl. No.: 19/153,805

(22) PCT Filed: Oct. 9, 2024
(86) PCT No.: PCT/CN2024/123529

§ 371 Date: Aug. 5, 2025

(30) Foreign Application Priority Data
Oct. 24, 2023 (CN) 202311382609.5

(51) Int. Cl. A61K 31/216; A61K 9/48

(52) U.S. Cl. CPC A61K 31/216; A61K 9/4875

(57) ABSTRACT

Provided is a fenofibrate-based oily formulation, an oral medicine, and a preparation method thereof, belonging to the technical field of pharmaceutical preparations. The fenofibrate-based oily formulation includes components of fenofibrate, an oily solvent, and a surfactant. A preparation method of the fenofibrate-based oily formulation is further provided, including the following steps: mixing the components, and then heating and stirring an obtained mixed product to obtain a clear oil solution as the fenofibrate-based oily formulation. The fenofibrate-based oily formulation enhances oral bioavailability of fenofibrate and ensures that oral absorption of the fenofibrate remains unaffected by dietary conditions.

### CROSS REFERENCE TO RELATED APPLICATION

This patent application is a national stage application of International Patent Application No. PCT/CN2024/123529, filed on Oct. 9, 2024, which claims the benefit and priority of Chinese Patent Application No. 202311382609.5 filed with the China National Intellectual Property Administration on Oct. 24, 2023, and entitled “FENOFIBRATE-BASED OILY FORMULATION, ORAL MEDICINE, AND PREPARATION METHOD THEREOF”, both of which are incorporated by reference herein in their entireties as part of the present application.

### TECHNICAL FIELD

The present disclosure belongs to the technical field of pharmaceutical preparations, and in particular relates to a fenofibrate-based oily formulation, an oral medicine, and preparation method thereof.

### BACKGROUND

Fenofibrate (isopropyl 2-methyl-2-[4-(4-chlorobenzoyl)phenoxy]propionate), a third-generation phenoxyacetic acid lipid-lowering agent, shares pharmacological mechanisms with clofibrate but has emerged as a preferred therapeutic option due to its potent lipid-lowering effects, fewer adverse reactions, and low long-term toxicity profile. As a prodrug, fenofibrate undergoes enzymatic hydrolysis by esterases following oral administration, with its metabolite fenofibric acid serving as the true active medicine responsible for pharmacological activity.

Fenofibrate is classified as a Biopharmaceutics Classification System (BCS) Class II drug, characterized by poor aqueous solubility and lipophilic properties. Currently marketed fenofibrate formulations globally primarily exist as capsules and tablets. The Lipanthyl® formulation employs micronization technology, where fenofibrate is processed with minimal surfactants via a jet-milling process to achieve a mixture with an average particle size of 6 μm to 7 μm. However, Lipanthyl® demonstrates significant variability in oral bioavailability of fenofibrate before or after meals, necessitating administration with meals—a requirement that substantially compromises patient compliance.

Current research efforts have focused on making improvements by addressing limitations of commercially available fenofibrate oral formulations, particularly their low bioavailability and poor patient compliance. However, most existing patents for fenofibrate oral formulations are predominantly limited to improving dissolution rates, while failing to demonstrate absorption advantages in animal models in vivo or only comparing bioavailability under fasted conditions. These approaches cannot confirm whether in vivo absorption of the formulation is unaffected by dietary condition.

Chinese Patent CN111759810B has described a preparation method of a fenofibrate nano-suspension based on solvent diffusion technology, utilizing anionic surfactants as stabilizers. This approach claims operational simplicity, cost-effectiveness, high reproducibility, and scalability for industrial production. While the nano-suspension demonstrates rapid dissolution of fenofibrate and enhanced gastrointestinal transport capacity. However, the patent documentation fails to provide comparative bioavailability data in vivo from animal studies, thereby limiting the evaluation of fenofibrate formulation advantages over existing products.

Chinese Patent CN109806240A has disclosed a method for preparing mesoporous carbon with adjustable particle sizes, along with the preparation of polymer-modified mesoporous carbon nanoparticles and use thereof as carriers for poorly soluble drugs to enhance oral absorption. The oral bioavailability of fenofibrate-loaded mesoporous carbon nanoparticles is reported to be 1.65 times that of commercial formulations. However, the preparation method for this formulation is notably complex, and the patent neither specifies the brand of the referenced commercial formulation nor clarifies whether administration occurred before or after meals.

Chinese Patent CN106727338B has disclosed a fenofibrate solid dispersion demonstrating excellent in vitro dissolution and in vivo bioavailability. Chinese Patent CN103599089B has described an oil-soluble sustained-release pharmaceutical formulation composition of fenofibrate, which exhibits favorable dissolution properties, high bioavailability, and controlled drug release for enhanced safety and reliability. However, the high bioavailability of both formulations is only demonstrated under fasted conditions, with a lack of animal experiments under fed conditions, thereby failing to establish superior oral absorption of fenofibrate compared to the commercial formulation Lipanthyl® in postprandial states.

Chinese Patent CN102309476B has presented a novel solid fenofibrate composition offering at least one of the following advantages: elimination or minimization of surfactant usage, superior characteristics such as enhanced bioavailability compared to existing products, and at least one improved attribute over liquid formulations (e.g., manufacturing processability, storage stability, ease of administration, and thermodynamic stability). Similar to Patent CN103599089B, the exemplary formulations only demonstrate a fasting-state increase in fenofibrate bioavailability to seven times that of Lipanthyl®, while still lacking comparative bioavailability data under fed conditions.

Numerous international patents have addressed the significant disparity in fenofibrate's oral bioavailability before and after meals. Although absorption of the disclosed fenofibrate oral formulations is not affected by dietary condition, their oral bioavailability remains suboptimal, coupled with complex preparation processes and requirements for advanced equipment.

U.S. Patent U.S. Ser. No. 07/041,319 has disclosed a fenofibrate nanoparticulate formulation unaffected by dietary conditions, corresponding to the commercially available product Tricor™ manufactured by an American company. The preparation of these fenofibrate nanoparticles requires substantial energy input, with potential stability degradation of active ingredients due to heating. The high free energy of the particles may induce particle aggregation, complicating the manufacturing process. Furthermore, the study “Katarina Bukara et al. In Vivo Performance of Fenofibrate Formulated With Ordered Mesoporous Silica Versus 2-Marketed Formulations: A Comparative Bioavailability Study in Beagle Dogs, Journal of Pharmaceutical Sciences, 2016, 105, 2381-2385” has compared Tricor™ with Lipanthyl®. Under fasted conditions in beagle dogs, a 48 mg dose of Tricor™ can yield an Area Under the Curve (AUC) of fenofibric acid of 11.80±1.170 ng/mL*h, representing 3.74 times that of Lipanthyl®. Nevertheless, the oral bioavailability of fenofibrate in this product remains suboptimal, indicating potential for further optimization.

South Korean Patent WO2022071768 has disclosed a fenofibrate pharmaceutical composition bioequivalent to Tricor™, which stabilizes micronized fenofibrate through surfactants and hydrophilic polymers, achieving favorable bioavailability and oral absorption unaffected by dietary condition. However, this patent requires micronized fenofibrate, imposing high demands on micronization equipment and relatively complex processes.

In summary, fenofibrate oral medicines demonstrating high bioavailability, absorption unaffected by dietary condition, simple preparation processes, and high stability hold significant market demand and promising development prospects.

### SUMMARY

An objective of the present disclosure is to provide a fenofibrate-based oily formulation, an oral medicine, and preparation method thereof. The fenofibrate-based oily formulation demonstrates high oral bioavailability, absorption unaffected by dietary condition, high preparation stability, and simple preparation process.

The present disclosure provides a fenofibrate-based oily formulation, including the following components in mass percentage: 0.5% to 20% of fenofibrate, 10% to 60% of an oily solvent, and 15% to 70% of a surfactant.

In some embodiments, the fenofibrate-based oily formulation includes the following components in mass percentage: 1% to 15% of the fenofibrate, 20% to 50% of the oily solvent, and 30% to 70% of the surfactant.

In some embodiments, the fenofibrate-based oily formulation includes the following components in mass percentage: 3% to 12% of the fenofibrate, 30% to 50% of the oily solvent, and 50% to 70% of the surfactant.

In some embodiments, the fenofibrate-based oily formulation further includes a co-surfactant in a mass percentage of 0% to 30%.

In some embodiments, the oily solvent is at least one or a commercial mixture containing at least one ingredient selected from the group consisting of soybean oil, rapeseed oil, palm oil, peanut oil, sunflower seed oil, coconut oil, flaxseed oil, castor oil, perilla oil, corn oil, glyceryl monostearate, glyceryl distearate, glyceryl tristearate, glyceryl monolinoleate, glyceryl dilinoleate, glyceryl trilinoleate, glyceryl monooleate, glyceryl dioleate, glyceryl trioleate, medium-chain triglycerides of varying chain lengths, a long-chain monoglyceride, a long-chain diglyceride, a long-chain triglyceride, caprylic/capric triglyceride, glyceryl trimyristate, isopropyl myristate, oleic acid, linoleic acid, ethyl oleate, ethyl linoleate, and palmitic acid.

In some embodiments, the surfactant is at least one or a commercial mixture containing at least one ingredient selected from the group consisting of polyoxyethylated castor oil, hydrogenated polyoxyethylated castor oil, Polyoxyethylene (20) sorbitan monolaurate (Tween™ 20), Polyoxyethylene (20) sorbitan monostearate (Tween™ 60), Polyoxyethylene (20) sorbitan monooleate (Tween™ 80), Vitamin E-tocopherol polyethylene glycol succinate (TPGS), polyethylene glycol glyceryl caprylate/caprate, lauroyl macrogolglycerides (32), polyethylene glycol (PEG)-32 stearate, and stearyl polyoxyethylene glyceride.

In some embodiments, the co-surfactant is at least one or a commercial mixture containing at least one ingredient selected from the group consisting of diethylene glycol monoethyl ether, polyglyceryl oleate, PEG400, isopropyl alcohol, propylene glycol laurate, ethanol, and 1,2-propylene glycol.

The present disclosure further provides a preparation method of the fenofibrate-based oily formulation, including the following steps: mixing the components, and then heating and stirring an obtained mixed product to obtain a clear oil solution as the fenofibrate-based oily formulation.

The present disclosure further provides an oral medicine, including the fenofibrate-based oily formulation.

In some embodiments, a dosage form of the oral medicine is selected from the group consisting of a soft capsule, a liquid capsule, a hard capsule, and a dropping pill.

Beneficial Effects: The present disclosure provides a fenofibrate-based oily formulation, including fenofibrate, an oily solvent, and a surfactant. Leveraging the characteristic of existing fenofibrate formulations exhibiting significantly increased postprandial bioavailability, the formulation facilitates lymphatic absorption and bypasses hepatic first-pass metabolism by promoting the binding of fenofibrate with chylomicrons through co-administration with oily media, thereby enhancing oral bioavailability and reducing effects by dietary condition on absorption.

Pharmacokinetic studies in rats and beagle dogs within examples of the present disclosure demonstrate that the formulation enhances the oral bioavailability of fenofibrate while exhibiting minimal effects by dietary condition on absorption. Dose conversion have revealed that in rats, the AUC of fenofibric acid following postprandial administration of Lipanthyl® is 196.52% of the fasting-state value, indicating a great change in bioavailability. In contrast, the AUC of fenofibric acid after postprandial administration of the self-emulsifying fenofibrate formulation (FNB-SEDDS) reaches 120.64% of the fasting-state level, reflecting a relative small improvement in bioavailability. In beagle dogs, the AUC of fenofibric acid following postprandial Lipanthyl® administration is 910.97% of the fasting-state value, indicating that as the animal size increases, the effects by dietary condition on the commercially available formulation of fenofibrate Lipanthyl® becomes more significant. The AUC of fenofibric acid after postprandial FNB-SEDDS administration in beagle dogs is 81.03% of the fasting-state level, showing no significant difference. The dual animal models confirm that the oil-based formulation herein achieves elevated oral bioavailability of fenofibrate while maintaining absorption unaffected by dietary condition.

### BRIEF DESCRIPTION OF THE DRAWINGS

FIG. 1 shows the plasma concentration-time curves of fenofibric acid following oral administration of Lipanthyl® and FNB-SEDDS in rats under fasted conditions;

FIG. 2 shows the plasma concentration-time curves of fenofibric acid following oral administration of Lipanthyl® and FNB-SEDDS in rats under fed conditions;

FIG. 3 shows the plasma concentration-time curves of fenofibric acid following oral administration of Lipanthyl® and FNB-SEDDS in beagle dogs under fasted conditions;

FIG. 4 shows the plasma concentration-time curves of fenofibric acid following oral administration of Lipanthyl® and FNB-SEDDS in beagle dogs along with meal.

### DETAILED DESCRIPTION OF THE EMBODIMENTS

The present disclosure provides a fenofibrate-based oily formulation, including the following components in mass percentage: 0.5% to 20% of fenofibrate, 10% to 60% of an oily solvent, and 15% to 70% of a surfactant.

In the present disclosure, the fenofibrate-based oily formulation includes fenofibrate, in some embodiments, the fenofibrate is at a mass content of 1% to 15%, and in some embodiments, the fenofibrate is at a mass content of 3% to 12%. The fenofibrate is chemically designated as isopropyl 2-methyl-2-[4-(4-chlorobenzoyl)phenoxy]propanoate, with a molecular formula of C₂₀H₂₁ClO₄, and its structure is represented by Formula I:

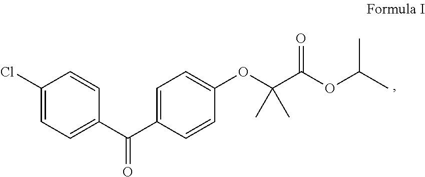 [SMILES: CC(C)OC(=O)C(C)(C)Oc1ccc(C(=O)c2ccc(Cl)cc2)cc1]

In the present disclosure, the fenofibrate-based oily formulation includes an oily solvent, in some embodiments, the oily solvent is at a mass percentage of 20% to 50%, and in some embodiments, the oily solvent is at a mass percentage of 30% to 50%. In some embodiments, the oily solvent is at least one or a commercial mixture containing at least one ingredient selected from the group consisting of soybean oil, rapeseed oil, palm oil, peanut oil, sunflower oil, coconut oil, flaxseed oil, castor oil, perilla oil, corn oil, glyceryl monostearate, glyceryl distearate, glyceryl tristearate, glyceryl monolinoleate, glyceryl dilinoleate, glyceryl trilinoleate, glyceryl monooleate, glyceryl dioleate, glyceryl trioleate, medium-chain (C6-C12) triglycerides of varying chain lengths, long-chain (C16-C22) monoglyceride, long-chain (C16-C22) diglyceride, long-chain (C16-C22) triglyceride, caprylic/capric triglyceride, glyceryl trimyristate, isopropyl myristate, oleic acid, linoleic acid, ethyl oleate, ethyl linoleate, and palmitic acid; in some embodiments, the oily solvent is at least one or a commercial mixture containing at least one ingredient selected from the group consisting of: oleic acid, linoleic acid, ethyl oleate, ethyl linoleate, glyceryl monolinoleate, glyceryl dilinoleate, glyceryl trilinoleate, glyceryl monooleate, glyceryl dioleate, glyceryl trioleate, medium-chain triglycerides of varying lengths, long-chain monoglyceride, long-chain diglyceride, long-chain triglyceride, and caprylic/capric triglyceride; and in some embodiments, the oily solvent is at least one or a commercial mixture containing at least one ingredient selected from the group consisting of: oleic acid, linoleic acid, ethyl oleate, ethyl linoleate, glyceryl monolinoleate, glyceryl dilinoleate, glyceryl trilinoleate, glyceryl monooleate, glyceryl dioleate, glyceryl trioleate, medium-chain triglycerides of varying lengths, and caprylic/capric triglyceride. The oily solvent enhances drug dispersion in the gastrointestinal tract, thereby improving oral bioavailability. Additionally, while dietary triglycerides promote lymphatic absorption of lipophilic fenofibrate, rendering its absorption pathway affected by dietary condition. The oily solvent in this formulation similarly facilitates lymphatic absorption of drug, thereby reducing the effects by dietary condition on fenofibrate's oral bioavailability.

In the present disclosure, the fenofibrate-based oily formulation includes a surfactant, in some embodiment, the surfactant is at a mass percentage of 30% to 70%, and in some embodiment, the surfactant is at a mass percentage of 50% to 70%. In some embodiments, the surfactant is at least one or a commercial mixture containing at least one ingredient selected from the group consisting of polyoxyethylated castor oil, hydrogenated polyoxyethylated castor oil, Tween™ 20, Tween™ 60, Tween™ 80, Vitamin E-TPGS, polyethylene glycol glyceryl caprylate/caprate, lauroyl macrogolglycerides(32), PEG-32 stearate, and stearyl polyoxyethylene glyceride; in some embodiments, the surfactant is at least one or a commercial mixture containing at least one ingredient selected from the group consisting of: polyoxyethylated castor oil, Tween™ 20, Vitamin E-TPGS, polyethylene glycol glyceryl caprylate/caprate, lauroyl macrogolglycerides(32), PEG-32 stearate, and stearyl polyoxyethylene glyceride; and in some embodiments, the surfactant is at least one or a commercial mixture containing at least one ingredient selected from the group consisting of: polyoxyethylated castor oil, Tween™ 20, Vitamin E-TPGS, polyethylene glycol glyceryl caprylate/caprate, and lauroyl macrogolglycerides(32). The surfactant functions to reduce surface tension and form monolayers at the oil-water interface, maintaining fenofibrate in a soluble state within the gastrointestinal tract, thereby enhancing intestinal epithelial absorption of the drug.

In the present disclosure, the fenofibrate-based oily formulation further includes a co-surfactant, with a mass percentage of 0% to 30%, in some embodiments, the fenofibrate-based oily formulation further includes a co-surfactant, with a mass percentage of 0% to 15%, and in some other embodiments, the fenofibrate-based oily formulation further includes a co-surfactant, with a mass percentage of 0% to 5%. In some embodiments, the co-surfactant is at least one or a commercial mixture containing at least one ingredient selected from the group consisting of diethylene glycol monoethyl ether, polyglyceryl oleate, PEG400, isopropyl alcohol, propylene glycol laurate, ethanol, and 1,2-propylene glycol; in some embodiments, the co-surfactant is at least one or a commercial mixture containing at least one ingredient selected from the group consisting of: diethylene glycol monoethyl ether, polyglyceryl oleate, and 1,2-propylene glycol. The co-surfactant enhances drug-loading capacity of the formulation and improves dispersion of drug in the formulation by increasing interfacial fluidity.

The present disclosure further provides a preparation method of the fenofibrate-based oily formulation, including the following steps: mixing the components, and then heating and stirring an obtained mixed product to obtain a clear oil solution as the fenofibrate-based oily formulation.

In some embodiments of the present disclosure, the mixed product is heated to 30° C. to 80° C., in some embodiments, the mixed product is heated to 30° C. to 60° C., and in some embodiments, the mixed product is heated to 35° C. to 40° C. The mixed product is stirred uniformly under the heating, and ultrasonic treatment at a frequency of 20 Hz to 100 Hz can be conducted to assist dissolution during stirring.

The present disclosure further provides an oral medicine, including the fenofibrate-based oily formulation.

In the present disclosure, a dosage form of the oral medicine is selected from the group consisting of a soft capsule, a liquid capsule, a hard capsule, and a dropping pill. There is no particular limitation on a preparation method of the oral medicine in the dosage form, and the oral medicine that meets the standards can be prepared using conventional technical means in the art.

To further illustrate the present disclosure, the fenofibrate-based oily formulation, the oral medicine, and the preparation method thereof provided in the present disclosure are described in detail below with reference to examples, but the examples should not be interpreted as a limitation to the protection scope of the present disclosure.

### Example 1

Components and weight percentages of the fenofibrate-based oily formulation were:

5.0% of fenofibrate, 47.5% of ethyl oleate, 38.0% of polyoxyethylated castor oil and 9.5% of Plurol Oleique CC497 (polyglyceryl oleate).

Preparation process for the fenofibrate-based oily formulation in this example included: 50 mg of fenofibrate, 475 mg of ethyl oleate, 380 mg of polyoxyethylated castor oil, and 95 mg of Plurol Oleique CC497 were added into a round-bottom flask, and a resulting mixed product was magnetically stirred at 40° C. for 30 min to obtain the fenofibrate-based oily formulation.

### Example 2

Components and weight percentages of the fenofibrate-based oily formulation were:

4.0% of fenofibrate, 38.4% of caprylic/capric triglyceride, 52.8% of Tween™20 and 4.8% 1,2-propylene glycol.

Preparation process for the fenofibrate-based oily formulation in this example included: 40 mg of fenofibrate, 384 mg of caprylic/capric triglyceride, 528 mg of Tween™ 20, and 48 mg of 1,2-propylene glycol were added into a round-bottom flask, and a resulting mixed product was magnetically stirred at 40° C. for 30 min to obtain the fenofibrate-based oily formulation.

### Example 3

Components and weight percentages of the fenofibrate-based oily formulation were:

4.0% of fenofibrate, 19.2% of Maisine CC (glyceryl monolinoleate), 67.2% of Tween™ 80 and 9.6% of Transcutol HP (diethylene glycol monoethyl ether).

Preparation process for the fenofibrate-based oily formulation in this example included: 40.0 mg of fenofibrate, 192.0 mg of Maisine CC, 672.0 mg of Tween™ 80, and 96.0 mg of Transcutol HP were added into a round-bottom flask, and a resulting mixed product was magnetically stirred at 40° C. for 30 min to obtain the fenofibrate-based oily formulation.

### Example 4

Components and weight percentages of the fenofibrate-based oily formulation were:

6.0% of fenofibrate, 18.8% of oleic acid, 61.1% of Tween™ 60 and 14.1% of vitamin E-TPGS.

Preparation process for the fenofibrate-based oily formulation in this example included: 60.0 mg of fenofibrate, 188.0 mg of oleic acid, 611.0 mg of Tween™ 60, and 141.0 mg of vitamin E-TPGS were added into a round-bottom flask, and a resulting mixed product was magnetically stirred at 40° C. for 30 min to obtain the fenofibrate-based oily formulation.

### Example 5

Components and weight percentages of the fenofibrate-based oily formulation were:

5.0% of fenofibrate, 28.5% of ethyl linoleate, 61.7% of Tween™ 20 and 4.8% of Labrasol (polyethylene glycol glyceryl caprylate/caprate).

Preparation process for the fenofibrate-based oily formulation in this example included: 50.0 mg of fenofibrate, 285.0 mg of ethyl linoleate, 617.0 mg of Tween™ 20, and 48.0 mg of Labrasol were added into a round-bottom flask, and a resulting mixed product was magnetically stirred at 40° C. for 30 min to obtain the fenofibrate-based oily formulation.

### Example 6

Components and weight percentages of the fenofibrate-based oily formulation were:

5.0% of fenofibrate, 19.0% of glyceryl trioleate, 61.7% of hydrogenated polyoxyethylated castor oil and 14.3% of PEG400.

Preparation process for the fenofibrate-based oily formulation in this example included: 50.0 mg of fenofibrate, 190.0 mg of glyceryl trioleate, 617.0 mg of hydrogenated polyoxyethylated castor oil, and 143.0 mg of PEG400 were added into a round-bottom flask, a resulting mixed product was magnetically stirred at 45° C. for 30 min to obtain the fenofibrate-based oily formulation.

### Example 7

Components and weight percentages of the fenofibrate-based oily formulation were:

5.0% of fenofibrate, 38.0% of Peceol (glyceryl monooleate), 52.3% of hydrogenated polyoxyethylated castor oil and 4.7% of Transcutol® HP.

Preparation process for the fenofibrate-based oily formulation in this example included: 50.0 mg of fenofibrate, 380.0 mg of Peceol, 523.0 mg of hydrogenated polyoxyethylated castor oil, and 47.0 mg of Transcutol® HP were added into a round-bottom flask, and a resulting mixed product was magnetically stirred at 40° C. for 30 min to obtain the fenofibrate-based oily formulation.

### Example 8

Components and weight percentages of the fenofibrate-based oily formulation were:

5.0% of fenofibrate, 28.5% of medium-chain triglyceride and 66.5% of Labrasol.

Preparation process for the fenofibrate-based oily formulation in this example included: 50.0 mg of fenofibrate, 285.0 mg of medium-chain triglyceride, and 665.0 mg of Labrasol were added into a round-bottom flask, and a resulting mixed product was magnetically stirred at 40° C. for 30 min to obtain the fenofibrate-based oily formulation.

### Example 9

Components and weight percentages of the fenofibrate-based oily formulation were:

6.0% of fenofibrate, 14.1% of glyceryl tristearate, 14.1% of glyceryl dioleate, 61.1% of hydrogenated polyoxyethylated castor oil and 4.7% of Transcutol® HP.

Preparation process for the fenofibrate-based oily formulation in this example included: 60.0 mg of fenofibrate, 141.0 mg of glyceryl tristearate, 141.0 mg of glyceryl dioleate, 611.0 mg of hydrogenated polyoxyethylated castor oil, and 47.0 mg of Transcutol® HP were added into a round-bottom flask, and a resulting mixed product was ultrasonicated at 45° C. and 50 Hz for 40 min to obtain the fenofibrate-based oily formulation.

### Example 10

Components and weight percentages of the fenofibrate-based oily formulation were:

7.0% of fenofibrate, 13.95% of glyceryl monooleate, 13.95% of glyceryl monostearate, 51.15% of Tween™ 20 and 13.95% of Transcutol® HP.

Preparation process for the fenofibrate-based oily formulation in this example included: 70.0 mg of fenofibrate, 139.5 mg of glyceryl monooleate, 139.5 mg of glyceryl monostearate, 511.5 mg of Tween™ 20, and 139.5 mg of Transcutol® HP were added into a round-bottom flask, and a resulting mixed product was ultrasonicated at 50° C. and 50 Hz for 40 min to obtain the fenofibrate-based oily formulation.

### Example 11

Components and weight percentages of the fenofibrate-based oily formulation were:

7.0% of fenofibrate, 13.95% of glyceryl dilinoleate, 13.95% of glyceryl distearate, 51.15% of Tween™ 20 and 13.95% of Transcutol® HP.

Preparation process for the fenofibrate-based oily formulation in this example included: 70.0 mg of fenofibrate, 139.5 mg of glyceryl dilinoleate, 139.5 mg of glyceryl distearate, 511.5 mg of Tween™ 20, and 139.5 mg of Transcutol® HP were added into a round-bottom flask, and a resulting mixed product was ultrasonicated at 50° C. and 50 Hz for 40 min to obtain the fenofibrate-based oily formulation.

### Example 12

Components and weight percentages of the fenofibrate-based oily formulation were:

7.0% of fenofibrate, 13.95% of glyceryl trilinoleate, 13.95% of glyceryl tristearate, 51.15% of Tween™ 20 and 13.95% of Transcutol® HP.

Preparation process for the fenofibrate-based oily formulation in this example included: 70.0 mg of fenofibrate, 139.5 mg of glyceryl trilinoleate, 139.5 mg of glyceryl tristearate, Tween™ 20, and 139.5 mg of Transcutol® HP were added into a round-bottom flask, and a resulting mixed product was ultrasonicated at 50° C. and 50 Hz for 40 min to obtain the fenofibrate-based oily formulation.

### Example 13

Components and weight percentages of the fenofibrate-based oily formulation were:

7.0% of fenofibrate, 13.95% of glyceryl trilinoleate, 13.95% of glyceryl tristearate, 51.15% of Tween™ 20, Lauroglycol™ 90 (propylene glycol laurate) 13.95%.

Preparation process for the fenofibrate-based oily formulation in this example included: 70.0 mg of fenofibrate, 139.5 mg of glyceryl trilinoleate, 139.5 mg of glyceryl tristearate, 511.5 mg of Tween™ 20, and 139.5 mg of Lauroglycol™ 90 were added into a round-bottom flask, and a resulting mixed product was ultrasonicated at 50° C. and 50 Hz for 40 min to obtain the fenofibrate-based oily formulation.

### Example 14

Components and weight percentages of the fenofibrate-based oily formulation were:

6.0% of fenofibrate, 28.2% of glyceryl trilinoleate, 28.2% of Tween™ 20, 28.2% of PEG-32 stearate, 9.4% of Lauroglycol™ 90 (propylene glycol laurate).

Preparation process for the fenofibrate-based oily formulation in this example included: 70.0 mg of fenofibrate, 282.0 mg of glyceryl trilinoleate, 282.0 mg of Tween™ 20, 282.0 mg of PEG-32 stearate, and 94.0 mg of Lauroglycol™ 90 were added into a round-bottom flask, and a resulting mixed product was ultrasonicated at 50° C. and 50 Hz for 40 min to obtain the fenofibrate-based oily formulation.

### Example 15

Components and weight percentages of the fenofibrate-based oily formulation were:

6.0% of fenofibrate, 28.2% of glyceryl trilinoleate, 28.2% of Tween™ 20, 28.2% of stearyl polyoxyethylene glyceride and 9.4% of Lauroglycol™ 90 (propylene glycol laurate).

Preparation process for the fenofibrate-based oily formulation in this example included: 70.0 mg of fenofibrate, 282.0 mg of glyceryl trilinoleate, 282.0 mg of Tween™ 20, 282.0 mg of stearyl polyoxyethylene glyceride, and 94.0 mg of Lauroglycol™ 90 were added into a round-bottom flask, and a resulting mixed product was ultrasonicated at 55° C. and 50 Hz for 40 min to obtain the fenofibrate-based oily formulation.

### Example 16

Components and weight percentages of the fenofibrate-based oily formulation were:

6.0% of fenofibrate, 28.2% of glyceryl trilinoleate, 28.2% of Tween™ 20, 28.2% of lauroyl macrogolglycerides(32) and 9.4% of Lauroglycol™ 90 (propylene glycol laurate).

Preparation process for the fenofibrate-based oily formulation in this example included: 70.0 mg of fenofibrate, 282.0 mg of glyceryl trilinoleate, 282.0 mg of Tween™ 20, 282.0 mg of lauroyl macrogolglycerides(32), and 94.0 mg of Lauroglycol™ 90 were added into a round-bottom flask, and a resulting mixed product was ultrasonicated at 55° C. and 50 Hz for 40 min to obtain the fenofibrate-based oily formulation.

### Experimental Example 1

Oral Bioavailability Evaluation of FNB-SEDDS (Prepared in Example 2) in Rats

20 healthy male Sprague-Dawley (SD) rats were randomly divided into 4 groups. Groups 1 and 2 were fasted for 12 h and then administered Lipanthyl® or FNB-SEDDS (both being 25 mg/kg) via intragastric administration, respectively. Blood samples were collected from the retro-orbital plexus under anesthesia at 0.5, 1, 1.5, 2, 4, 8, 12, and 24 h post-dosing.

Groups 3 and 4 were administered Lipanthyl® or FNB-SEDDS (both being 16.7 mg/kg) via intragastric administration under fed conditions, respectively. Blood samples were collected at 0.5, 1, 1.5, 2, 4, 6, 8, 12, and 24 h post-dosing. Plasma concentrations were measured at designated time points, and relative bioavailability was calculated based on the area under the curve (AUC) using established methods for relative bioavailability studies.

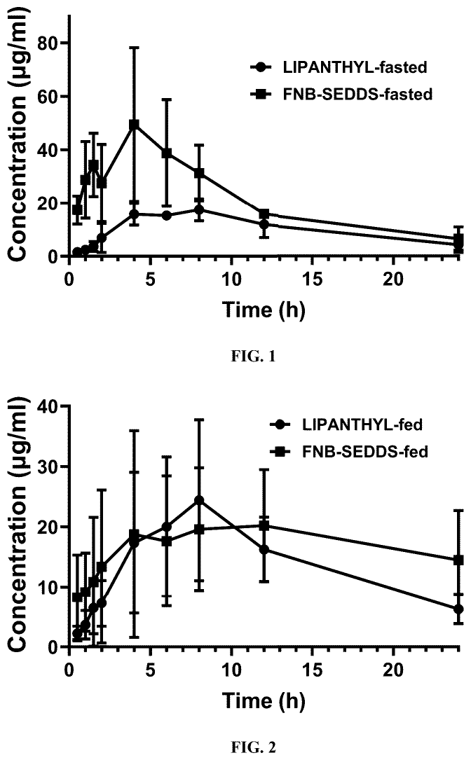

The results were shown in FIG. 1 and FIG. 2. Under fasted conditions, the AUC of fenofibric acid after Lipanthyl® administration in rats was 249.7±39.7 μg/mL·h, while the AUC after FNB-SEDDS administration reached 512.6±103.8 μg/mL·h, corresponding to a relative oral bioavailability of 205.29% for FNB-SEDDS compared to Lipanthyl® (FIG. 1). Under fed conditions, the AUC of fenofibric acid after Lipanthyl® administration was 327.80±68.10 μg/mL·h, whereas the AUC after FNB-SEDDS administration was 413.08±104.14 μg/mL·h, yielding a relative oral bioavailability of 126.02% for FNB-SEDDS versus Lipanthyl® (FIG. 2). Detailed pharmacokinetic parameters were summarized in Table 1. These findings demonstrated that the FNB-SEDDS formulation of the present disclosure significantly enhanced the oral bioavailability of fenofibrate.

Dose conversion had revealed that in rats, the AUC of fenofibric acid following postprandial administration of Lipanthyl® was 196.52% of the fasting-state value, indicating a great change in bioavailability. However, there was no statistical difference in the AUC of rats after administration of FNB-SEDDS in the fed state and the fasted state. These findings demonstrated that the oral bioavailability of fenofibrate in FNB-SEDDS was not affected by dietary condition.

TABLE 1
Pharmacokinetic parameters of fenofibric acid in rats after oral administration
of Lipanthyl ® and FNB-SEDDS under fasted/fed conditions

| | Lipanthyl ®-fasted | Lipanthyl ®-fed | FNB-SEDDS-fasted | FNB-SEDDS-fed |
|---|---|---|---|---|
| Administration | 25 mg/kg | 16.7 mg/kg | 25 mg/kg | 16.7 mg/kg |
| dose | | | | |
| AUC₍₀₋ₜ₎(μg/mL*h) | 249.7 ± 39.7 | 327.80 ± 68.10*** | 512.6 ± 103.8 | 413.08 ± 104.14 |
| Cₘₐₓ(μg/mL) | 19.66 ± 2.58 | 29.965 ± 9.09*** | 59.24 ± 18.22 | 31.46 ± 7.93 |
| t_{1/2}(h) | 9.28 ± 7.44 | 19.06 ± 20.54 | 15.39 ± 20.145 | 14.57 ± 17.32 |
| Tₘₐₓ(h) | 6.00 ± 2.04 | 6.40 ± 1.67 | 3.00 ± 1.37 | 10.8 ± 8.67 |
| Relative | | | 205.29 (vs. | 126.02 (vs. |
| bioavailability (%) | | | Lipanthyl ®-fasted) | Lipanthyl ®-fed) |

In Table 1,
***p < 0.001 compared with the Lipanthyl ®-fasted.

### Experimental Example 2

### Oral Bioavailability Evaluation of FNB-SEDDS (Prepared in Example 3) in Beagle Dogs

The study involved 6 male beagle dogs divided into two groups in a crossover design with a 1-week washout period, conducted over four weeks. During Weeks 1 and 2, dogs were fasted (with free access to water) for 12 h before administration: one group received 2 FNB-SEDDS enteric-coated capsules (144 mg fenofibrate), and the other received 1 Lipanthyl® capsule (200 mg fenofibrate). In Weeks 3 and 4, under fed conditions following a 12-h fast (with water), the same groups were administered 2 FNB-SEDDS enteric-coated capsules (144 mg fenofibrate) or 1 Lipanthyl® capsule (200 mg fenofibrate), respectively. Blood samples were collected from the forelimb vein at 0.5, 1, 2, 4, 6, 8, 12, and 24 h post-dosing. Plasma concentrations of fenofibric acid were measured at designated time points, and relative bioavailability was calculated based on the area under the curve (AUC) using established methods for relative bioavailability studies.

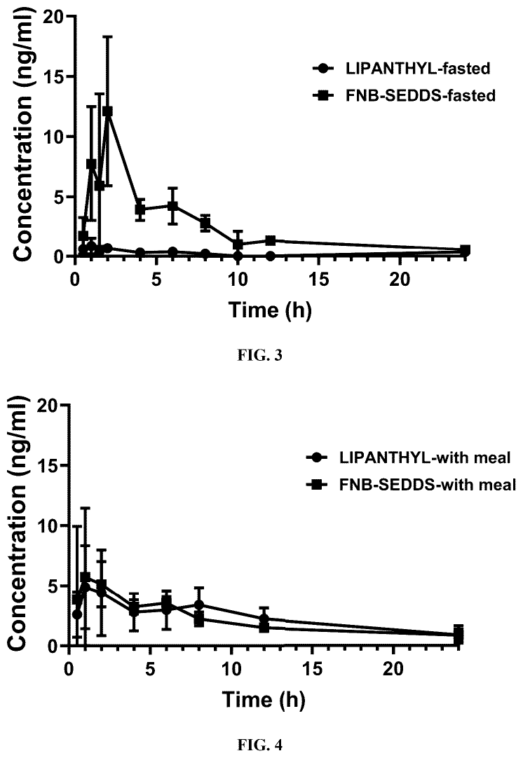

Under fasted conditions, the AUC of fenofibric acid following Lipanthyl® administration in beagle dogs was 6.25±3.39 μg/mL·h, while the AUC after FNB-SEDDS administration was 63.88±17.20 μg/mL·h. After dose conversion, relative bioavailability of fenofibrate for the FNB-SEDDS reached 1418.93% compared to Lipanthyl® (FIG. 3). Under fed conditions, the AUC of fenofibric acid after Lipanthyl® administration was 56.96±15.31 μg/mL·h, whereas the AUC after FNB-SEDDS administration was 51.76±9.59 μg/mL·h. After dose conversion, bioavailability of fenofibrate for the FNB-SEDDS was 126.21% compared to Lipanthyl® (FIG. 4). Detailed pharmacokinetic parameters were provided in Table 2. The beagle dog experiments confirmed that the FNB-SEDDS formulation of the present disclosure enhanced the oral absorption of fenofibrate.

In beagle dogs, the AUC of fenofibric acid following postprandial Lipanthyl® administration has surged to 910.97% of the fasting-state value, indicating that as the animal size increased, the effects by dietary condition on the commercially available formulation of fenofibrate Lipanthyl® become more significant. The AUC of fenofibric acid following FNB-SEDDS administration in beagle dogs under fed conditions showed no significant difference compared to AUC under fasting-state. The dual animal models confirmed that the oral formulation herein achieved elevated oral bioavailability of fenofibrate while maintaining absorption unaffected by dietary condition.

TABLE 2
Pharmacokinetic parameters of fenofibric acid in beagle dogs after oral administration
of Lipanthyl ® and FNB-SEDDS under fasted/with meal conditions

| | | Lipanthyl ®-with | FNB-SEDDS- | FNB-SEDDS- |
|---|---|---|---|---|
| | Lipanthyl ®-fasted | meal | fasted | with meal |
| Administration | 200 mg/dog | 200 mg/dog | 144 mg/dog | 144 mg/dog |
| dose | | | | |
| AUC₍₀₋ₜ₎(μg/mL*h) | 6.25 ± 3.39 | 56.96 ± 15.31*** | 63.88 ± 17.200 | 51.76 ± 9.59 |
| Cₘₐₓ(μg/mL) | 1.20 ± 0.79 | 6.01 ± 2.68** | 14.47 ± 5.65 | 7.34 ± 4.54 |
| t_{1/2}(h) | 2.27 ± 1.19 | 15.00 ± 13.73 | 4.95 ± 1.58 | 9.78 ± 5.73 |
| Tₘₐₓ(h) | 5.00 ± 9.32 | 4.33 ± 3.39 | 1.83 ± 0.26 | 2.42 ± 2.80 |
| Relative | | | 1,418.93 (vs. | 126.21 (vs. |
| bioavailability (%) | | | Lipanthyl ®-fasted) | Lipanthyl ®-with |
| | | | | meal) |

**p < 0.01,
***p < 0.001 compared with Lipanthyl ®-fasted.

Although the present application has been described in detail through the above examples, the examples are merely some rather than all of the examples of the present application. All other examples obtained by a person based on these examples without creative efforts shall fall within the protection scope of the present application.

1. A fenofibrate-based oily formulation, comprising the following components in mass percentage: 0.5% to 20% of fenofibrate, 10% to 60% of an oily solvent, 15% to 70% of a surfactant, and 0% to 30% of a co-surfactant.

2. The fenofibrate-based oily formulation according to claim 1, comprising the following components in mass percentage: 1% to 15% of the fenofibrate, 20% to 50% of the oily solvent, 30% to 70% of the surfactant, and 0% to 15% of the co-surfactant.

3. The fenofibrate-based oily formulation according to claim 1, comprising the following components in mass percentage: 3% to 12% of the fenofibrate, 30% to 50% of the oily solvent, 50% to 67% of the surfactant, and 0% to 5% of the co-surfactant.

4. The fenofibrate-based oily formulation according to claim 1, wherein the oily solvent is at least one or a mixture comprising at least one ingredient selected from the group consisting of soybean oil, rapeseed oil, palm oil, peanut oil, sunflower seed oil, coconut oil, flaxseed oil, castor oil, perilla oil, corn oil, glyceryl monostearate, glyceryl distearate, glyceryl tristearate, glyceryl monolinoleate, glyceryl dilinoleate, glyceryl trilinoleate, glyceryl monooleate, glyceryl dioleate, glyceryl trioleate, medium-chain triglycerides of varying chain lengths, a long-chain monoglyceride, a long-chain diglyceride, a long-chain triglyceride, caprylic/capric triglyceride, glyceryl trimyristate, isopropyl myristate, oleic acid, linoleic acid, ethyl oleate, ethyl linoleate, and palmitic acid.

5. The fenofibrate-based oily formulation according to claim 1, wherein the surfactant is at least one or a commercial mixture comprising at least one ingredient selected from the group consisting of polyoxyethylated castor oil, hydrogenated polyoxyethylated castor oil, Tween™ 20, Tween™ 60, Tween™ 80, Vitamin E-tocopherol polyethylene glycol succinate (TPGS), polyethylene glycol glyceryl caprylate/caprate, lauroyl macrogolglycerides(32), polyethylene glycol (PEG)-32 stearate, and stearyl polyoxyethylene glyceride.

6. The fenofibrate-based oily formulation according to claim 1, wherein the co-surfactant is at least one or a commercial mixture comprising at least one ingredient selected from the group consisting of diethylene glycol monoethyl ether, polyglyceryl oleate, PEG400, isopropyl alcohol, propylene glycol laurate, ethanol, and 1,2-propylene glycol.

7. A method for preparing of the fenofibrate-based oily formulation according to claim 1, comprising the following steps: mixing the components, and then heating and stirring an obtained mixed product to obtain a clear oil solution as the fenofibrate-based oily formulation.

8. The method according to claim 7, wherein the mixed product is heated to 30° C. to 80° C.

9. The method according to claim 8, wherein the stirring is accompanied by an ultrasonic treatment at a frequency of 20 Hz to 100 Hz.

10. An oral medicine, comprising the fenofibrate-based oily formulation according to claim 1.

11. The oral medicine according to claim 10, wherein a dosage form of the oral medicine is selected from the group consisting of a soft capsule, a liquid capsule, a hard capsule, and a dropping pill.

12. The fenofibrate-based oily formulation according to claim 2, wherein the oily solvent is at least one or a mixture comprising at least one ingredient selected from the group consisting of soybean oil, rapeseed oil, palm oil, peanut oil, sunflower seed oil, coconut oil, flaxseed oil, castor oil, perilla oil, corn oil, glyceryl monostearate, glyceryl distearate, glyceryl tristearate, glyceryl monolinoleate, glyceryl dilinoleate, glyceryl trilinoleate, glyceryl monooleate, glyceryl dioleate, glyceryl trioleate, medium-chain triglycerides of varying chain lengths, a long-chain monoglyceride, a long-chain diglyceride, a long-chain triglyceride, caprylic/capric triglyceride, glyceryl trimyristate, isopropyl myristate, oleic acid, linoleic acid, ethyl oleate, ethyl linoleate, and palmitic acid.

13. The fenofibrate-based oily formulation according to claim 3, wherein the oily solvent is at least one or a mixture comprising at least one ingredient selected from the group consisting of soybean oil, rapeseed oil, palm oil, peanut oil, sunflower seed oil, coconut oil, flaxseed oil, castor oil, perilla oil, corn oil, glyceryl monostearate, glyceryl distearate, glyceryl tristearate, glyceryl monolinoleate, glyceryl dilinoleate, glyceryl trilinoleate, glyceryl monooleate, glyceryl dioleate, glyceryl trioleate, medium-chain triglycerides of varying chain lengths, a long-chain monoglyceride, a long-chain diglyceride, a long-chain triglyceride, caprylic/capric triglyceride, glyceryl trimyristate, isopropyl myristate, oleic acid, linoleic acid, ethyl oleate, ethyl linoleate, and palmitic acid.

14. The fenofibrate-based oily formulation according to claim 2, wherein the surfactant is at least one or a commercial mixture comprising at least one ingredient selected from the group consisting of polyoxyethylated castor oil, hydrogenated polyoxyethylated castor oil, Tween™ 20, Tween™ 60, Tween™ 80, Vitamin E-tocopherol polyethylene glycol succinate (TPGS), polyethylene glycol glyceryl caprylate/caprate, lauroyl macrogolglycerides(32), polyethylene glycol (PEG)-32 stearate, and stearyl polyoxyethylene glyceride.

15. The fenofibrate-based oily formulation according to claim 3, wherein the surfactant is at least one or a commercial mixture comprising at least one ingredient selected from the group consisting of polyoxyethylated castor oil, hydrogenated polyoxyethylated castor oil, Tween™ 20, Tween™ 60, Tween™ 80, Vitamin E-tocopherol polyethylene glycol succinate (TPGS), polyethylene glycol glyceryl caprylate/caprate, lauroyl macrogolglycerides(32), polyethylene glycol (PEG)-32 stearate, and stearyl polyoxyethylene glyceride.

16. The fenofibrate-based oily formulation according to claim 2, wherein the co-surfactant is at least one or a commercial mixture comprising at least one ingredient selected from the group consisting of diethylene glycol monoethyl ether, polyglyceryl oleate, PEG400, isopropyl alcohol, propylene glycol laurate, ethanol, and 1,2-propylene glycol.

17. The fenofibrate-based oily formulation according to claim 3, wherein the co-surfactant is at least one or a commercial mixture comprising at least one ingredient selected from the group consisting of diethylene glycol monoethyl ether, polyglyceryl oleate, PEG400, isopropyl alcohol, propylene glycol laurate, ethanol, and 1,2-propylene glycol.

18. A method for preparing the fenofibrate-based oily formulation according to claim 2, comprising the following steps: mixing the components, and then heating and stirring an obtained mixed product to obtain a clear oil solution as the fenofibrate-based oily formulation.

19. A method for preparing the fenofibrate-based oily formulation according to claim 3, comprising the following steps: mixing the components, and then heating and stirring an obtained mixed product to obtain a clear oil solution as the fenofibrate-based oily formulation.

20. An oral medicine, comprising the fenofibrate-based oily formulation according to claim 2.
